(19) United States
(12) Patent Application Publication
(10) Pub. No.: US 2023/0047456 A1
(43) Pub. Date: Feb. 16, 2023

(54) OIL-IN-WATER-TYPE EMULSIFIED COSMETIC

(71) Applicant: SHISEIDO COMPANY, LTD., Chuo-ku, Tokyo (JP)

(72) Inventors: Reina WATANABE, Tokyo (JP); Minori NAKAJIMA, Tokyo (JP); Haruka NISHI, Tokyo (JP)

(73) Assignee: SHISEIDO COMPANY, LTD., Chuo-ku, Tokyo (JP)

(21) Appl. No.: 17/788,582

(22) PCT Filed: Dec. 22, 2020
(86) PCT No.: PCT/JP2020/048022

§ 371 Date: Jun. 23, 2022

(30) Foreign Application Priority Data
Dec. 25, 2019 (JP) 2019-234484

(51) Int. Cl. A61K 8/06; A61Q 17/04; A61K 8/02

(52) U.S. Cl. CPC A61K 8/062; A61Q 17/04; A61K 8/0241; A61K 2800/413; A61K 2800/412; A61K 2800/624

(57) ABSTRACT

To provide an oil-in-water type emulsified cosmetic comprising a pigment grade hydrophobically treated particle and a hydrophobically treated microparticle, which is capable of uniformly applying particles subjected to hydrophobization treatment on the surface of the skin and exhibiting a good tone-up effect and the like.

The oil-in-water type emulsified cosmetic of the present disclosure comprises a dispersion medium comprising water, and an oil droplet dispersed in the dispersion medium, wherein the dispersion medium comprises a nonionic surfactant, a polyhydric alcohol, and a pigment grade hydrophobically treated particle having an average particle diameter of 300 nm or more, wherein the oil droplet comprises an oil content , a nonionic surfactant, and a hydrophobically treated microparticle having an average particle diameter of 200 nm or less, and wherein the oil content comprises a volatile oil.

### FIELD

The present disclosure relates to an oil-in-water type emulsified cosmetic.

### BACKGROUND

In the field of cosmetics, an oil-in-water type emulsified cosmetic containing particles subjected to hydrophobization treatment has been developed.

PTL 1 discloses an oil-in-water emulsification type sunscreen cosmetic comprising (A) an acrylic polymer; (B) an ultraviolet shielding agent composed of hydrophobically treated inorganic fine particles; (C) a nonionic surfactant having an HLB value of 6.5 or less and liquid or semi-solid form at 20° C.; and (D) a nonionic surfactant having an HLB value of 10 or more.

PTL 2 discloses an oil-in-water type emulsified cosmetic comprising (A) 0.05 to 1% by mass of a hydrophobically modified alkyl cellulose; (B) 5 to 40% by mass of an oil content; (C) 2.5 to 30% by mass of an ultraviolet scattering agent having a hydrophobic surface; and (D) an aqueous phase thickener having low salt resistance, wherein (C) the ultraviolet scattering agent is dispersed in an oil phase.

### CITATION LIST

### Patent Literature

[PTL 1] Japanese Unexamined Patent Publication (Kokai) No. 2019-123674

[PTL 2] Japanese Unexamined Patent Publication (Kokai) No. 2015-120682

### SUMMARY

### Technical Problem

In order to improve familiarity to the skin and the like, hydrophobization treatment may be applied to particles to be blended into an emulsified cosmetic. In an oil-in-water type emulsified cosmetic, the hydrophobically treated particles are generally blended into an oil phase (oil droplets).

When a cosmetic in such a state is applied to the skin, the oil droplets tend to adsorb near the crista cutis portion of the skin having high surface activity, while the adsorbed oil droplets containing the hydrophobically treated particles move to the sulcus cutis portion of the skin until the moisture dries, resulting in unevenness of the fine particles, and thus, there has been a case where a defect such as gathering and dropping of powder into sulcus cutis of the skin, or powder squeakiness occurs. For example, when relatively large hydrophobically treated pigment grade particles are used for the purpose of producing a tone-up effect of lightening the color of the skin, the tendency becomes more remarkable, and there has been a fear that a tone-up unevenness or the like may be caused by the dropped pigment grade particles.

Accordingly, it is a subject of the present disclosure to provide an oil-in-water type emulsified cosmetic comprising a pigment grade hydrophobically treated particle and a hydrophobically treated microparticle, which is capable of uniformly applying particles subjected to hydrophobization treatment on the surface of the skin and exhibiting a good tone-up effect and the like.

### Solution to Problem

### Embodiment 1

An oil-in-water type emulsified cosmetic comprising:

a dispersion medium comprising water and

an oil droplet dispersed in the dispersion medium,

wherein the dispersion medium comprises a nonionic surfactant, a polyhydric alcohol, and a pigment grade hydrophobically treated particle having an average particle diameter of 300 nm or more,

wherein the oil droplet comprises an oil content, a nonionic surfactant, and a hydrophobically treated microparticle having an average particle diameter of 200 nm or less, and

wherein the oil content comprises a volatile oil.

### Embodiment 2

The cosmetic according to embodiment 1, wherein the content of the oil content is 20% by mass or more.

### Embodiment 3

The cosmetic according to embodiment 1 or 2, wherein the volatile oil is contained in the oil content in an amount of 10% by mass or more.

### Embodiment 4

The cosmetic according to any of embodiments 1 to 3, wherein an HLB of the nonionic surfactant is 10.0 to 16.0.

### Embodiment 5

The cosmetic according to any of embodiments 1 to 4, wherein the nonionic surfactant is a polyether-modified silicone.

### Embodiment 6

The cosmetic according to embodiment 5, wherein the mass ratio of the pigment grade hydrophobically treated particle to the polyether-modified silicone having an HLB of 10.0 to 16.0 is 11 to 30.

### Embodiment 7

The cosmetic according to any one of embodiments 1 to 6, wherein the oil content comprises at least one selected from a polar oil and an ultraviolet absorber.

### Embodiment 8

The cosmetic according to embodiment 7, wherein the at least one selected from the polar oil and the ultraviolet absorber is contained in an amount of 10% by mass or more based on the total amount of the oil content.

### Embodiment 9

The cosmetic according to any one of embodiments 1 to 8, wherein the hydrophobically treated microparticle is an ultraviolet scattering agent.

### Advantageous Effects of Invention

According to the present disclosure, it is possible to provide an oil-in-water type emulsified cosmetic comprising a pigment grade hydrophobically treated particle and a hydrophobically treated microparticle, which is capable of uniformly applying particles subjected to hydrophobization treatment on the surface of the skin and exhibiting a good tone-up effect and the like.

### BRIEF DESCRIPTION OF DRAWINGS

FIG. 1 (a) is a schematic diagram of an oil-in-water type emulsified cosmetic of one embodiment of the present disclosure, and (b) is a schematic diagram immediately after applying this cosmetic to the skin, and (c) is a schematic diagram of a skin surface in a state in which the cosmetic is adapted to the skin after drying moisture, and (d) is a schematic diagram showing a state of the cosmetic after a further lapse of time from the state of (c) when the cosmetic does not contain a volatile oil as an oil content, and (e) is a schematic diagram showing a state of the cosmetic after a further lapse of time from the state of (c) when the cosmetic contains a volatile oil as an oil content.

FIG. 2 (a) is a schematic diagram of an oil-in-water type emulsified cosmetic containing hydrophilic pigment grade particles dispersed in an aqueous phase and oil droplets containing hydrophobically treated microparticles, (b) is a schematic diagram immediately after applying this cosmetic to the skin, and (c) is a schematic diagram of the skin surface after drying moisture.

FIG. 3 (a) is a schematic diagram of an oil-in-water type emulsified cosmetic comprising oil droplets containing pigment grade hydrophobically treated particles and hydrophobically treated microparticles, (b) is a schematic diagram immediately after applying this cosmetic to the skin, and (c) is a schematic diagram of the skin surface after drying moisture.

FIG. 4 (a) is a schematic diagram of an oil-in-water type emulsified cosmetic containing pigment grade hydrophobically treated particles and hydrophobically treated microparticles dispersed in an aqueous phase, (b) is a schematic diagram immediately after applying this cosmetic to the skin, and (c) is a schematic diagram of the skin surface after drying moisture.

### DESCRIPTION OF EMBODIMENTS

Hereinafter, embodiments of the present disclosure will be described in detail. The present disclosure is not limited to the following embodiments, and may be variously modified and practiced within the scope of the present invention.

In the oil-in-water type emulsified cosmetic of the present disclosure, a dispersion medium containing water contains a pigment grade hydrophobically treated particle having an average particle diameter of 300 nm or more, and an oil droplet dispersed in the dispersion medium contains an oil content, a nonionic surfactant, a polyhydric alcohol, and a hydrophobically treated microparticle having an average particle diameter of 200 nm or less, and the oil content contains a volatile oil.

Although not limited by the principle, it is considered that the action principle in which the cosmetic of the present disclosure can uniformly apply particles subjected to hydrophobization treatment to the surface of the skin and can exhibit a tone-up effect or the like is as follows.

When an emulsified cosmetic containing hydrophobically treated particles is obtained, it is general that hydrophobically treated particles are blended in an oil phase because of hydrophobicity. However, the present inventor has found that, among hydrophobically treated particles, when a pigment grade particle having a large size is blended on an aqueous phase side and a hydrophobically treated microparticle smaller than that is blended on an oil phase side and a volatile oil is used as an oil content, surprisingly, when an oil-in-water type emulsified cosmetic containing these particles is applied to the skin, these particles are uniformly applied to the surface of the skin, and a tone-up effect or the like is improved.

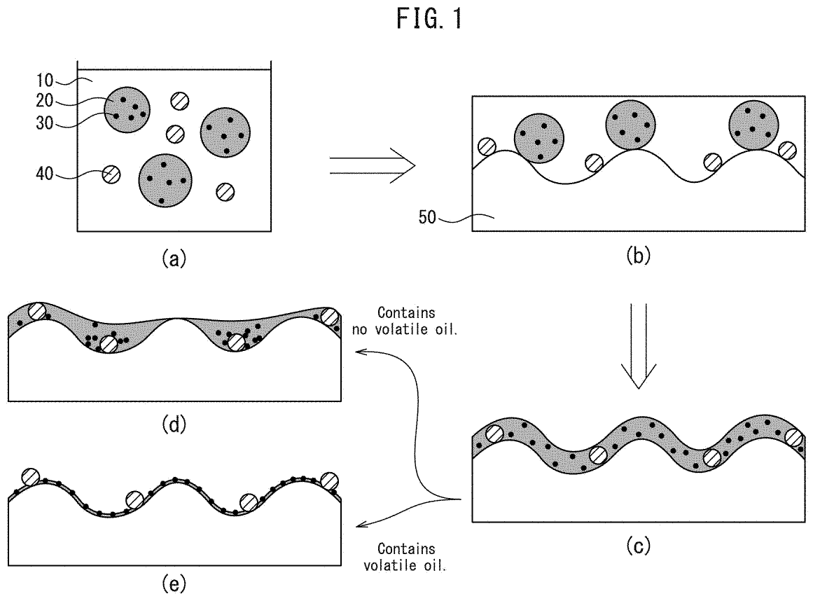

When the oil-in-water type emulsified cosmetic of the present disclosure is applied to the skin, as shown in (b) of FIG. 1, relatively large pigment grade hydrophobically treated particles 40 and oil droplets are adsorbed on the skin 50 which is generally hydrophobic. Thereafter, it is considered that the oil content 20 in the oil droplet moves in the direction of the sulcus cutis portion from the vicinity of the crista cutis portion until the moisture of the cosmetic dries. Since the hydrophobic pigment grade particles are easily familiar with the oil content, they are wrapped in the oil content without repelling the moved oil content. As a result, it is considered that an oil film containing uniformly the hydrophobically treated microparticles 30 and the pigment grade hydrophobically treated particles 40 as shown in (c) of FIG. 1 is uniformly formed on the surface of the skin 50.

However, even if such an oil film is temporarily formed, when the oil content 20 in the oil droplet does not contain a volatile oil, over time, the hydrophobically treated microparticles 30 and the pigment grade hydrophobically treated particles 40 can move in the oil film, and therefore, as shown in (d) of FIG. 1, in the sulcus cutis portion of the skin, etc., unevenness of the particles may occur, causing a defect such as gathering and dropping of powder into sulcus cutis of the skin, or powder squeakiness. On the other hand, when the oil content 20 in the oil droplet contains a volatile oil, over time, this volatile oil volatilizes and the amount of oil content in the oil film decreases, so that these particles hardly move through the oil film. As a result, it is considered that these particles can be uniformly adsorbed and stayed on the surface of the skin as shown in (e) of FIG. 1. In addition, when the oil content includes a volatile oil and a nonvolatile oil, it is considered that when the volatile oil volatilizes and the amount of oil content in the oil film decreases, the nonvolatile oil itself becomes attracted to the particles on the surface of the skin and can stay. As a result, it is considered that a uniform oil film is formed because the movement of the nonvolatile oil is suppressed.

Thus, the cosmetic of the present disclosure can satisfactorily exert each function, such as a tone-up function and an ultraviolet scattering function, provided by each particle of the pigment grade hydrophobically treated particle and the hydrophobically treated microparticle. In addition, since the cosmetic of the present disclosure can contain a nonvolatile and an organic ultraviolet absorber as an oil content in an oil film, the formation of a uniform oil film containing the ultraviolet absorber can also contribute to the improvement of the ultraviolet protective effect (SPF). It is possible to improve the SPF by dispersing the pigment grade hydrophobically treated particles in an outer phase (aqueous phase) rather than dispersing them in an inner phase (oil phase).

Further, even if the hydrophobically treated microparticles are coarsened by aggregation in the oil droplet, since the degree of coarsening is low as compared with aggregation between the pigment grade hydrophobically treated particles, it is considered that adverse effects such as color unevenness due to the aggregated hydrophobically treated microparticles hardly occur.

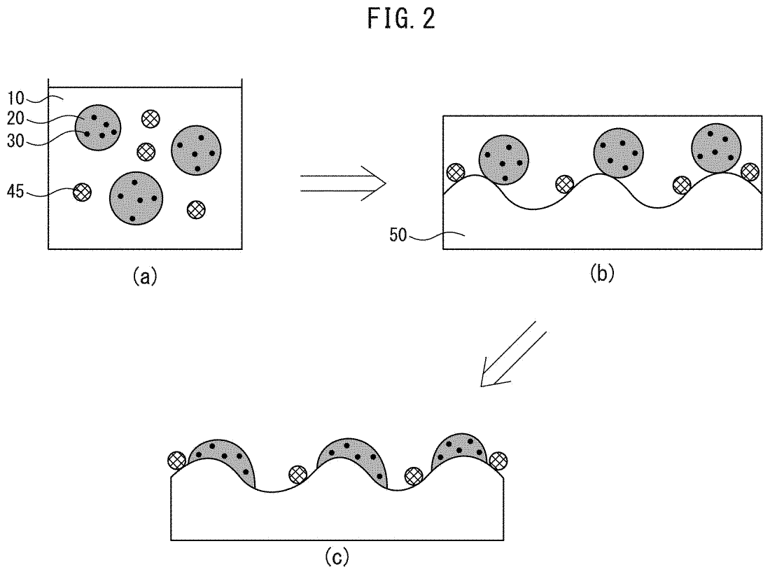

On the other hand, in the case of an emulsified cosmetic containing hydrophilic pigment grade particles 45 in an aqueous phase as shown in FIG. 2, since the hydrophilic pigment grade particles 45 tend to repel the oil content that has moved, it is considered difficult to form a uniform oil film containing the hydrophobically treated microparticles 30 and the hydrophilic pigment grade particles 45. Since the hydrophilic pigment grade particles are not applied to the skin surface together with the oil film in addition to being difficult to be familiar with the hydrophobic skin, it is considered that the particles tend to exhibit gathering and dropping of powder into sulcus cutis of the skin, or powder squeakiness. As a result, when such a cosmetic is applied to the skin surface, it is considered that a portion where the hydrophobically treated microparticles are locally present or a portion where the pigment grade particles fall off from the skin occurs, so that each function (for example, a tone-up function and an ultraviolet scattering function) provided by each particle of the hydrophobically treated microparticle and the pigment grade particle cannot be sufficiently exerted.

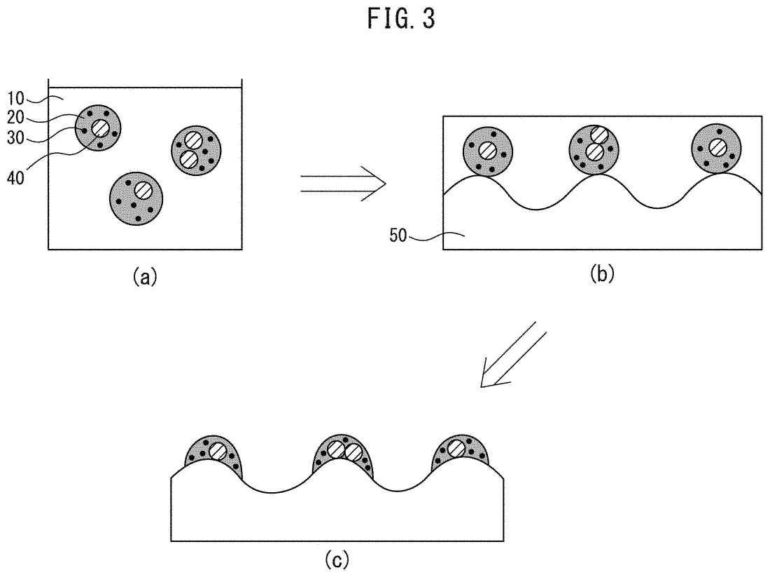

In the case of an emulsified cosmetic containing both of the hydrophobically treated microparticles 30 and the pigment grade hydrophobically treated particles 40 in an oil droplet as shown in FIG. 3, it is considered that the particles are more easily aggregated in the oil droplet. In particular, aggregation of pigment grade hydrophobically treated particles is likely to cause color unevenness or tone-up unevenness. Even if only the hydrophobically treated microparticles 30 of FIG. 3 are dispersed in place of the oil phase to the aqueous phase, since the pigment grade hydrophobically treated particles in this oil droplet tend to aggregate, it is considered that an emulsified cosmetic containing the hydrophobically treated microparticles in the aqueous phase and the pigment grade hydrophobically treated particles in the oil phase similarly tends to cause a defect such as color unevenness or tone-up unevenness.

In addition, when the hydrophobically treated microparticles and the pigment grade hydrophobically treated particles are contained in the oil droplets at the same time, even if the cosmetic containing such oil droplets is applied to the skin, the oil content is attracted by both particles which are hydrophobically, so that the oil content is not spread as in FIG. 1, and both particles are locally arranged on the skin surface, so that it is considered that each function exhibited by both particles cannot be sufficiently exerted.

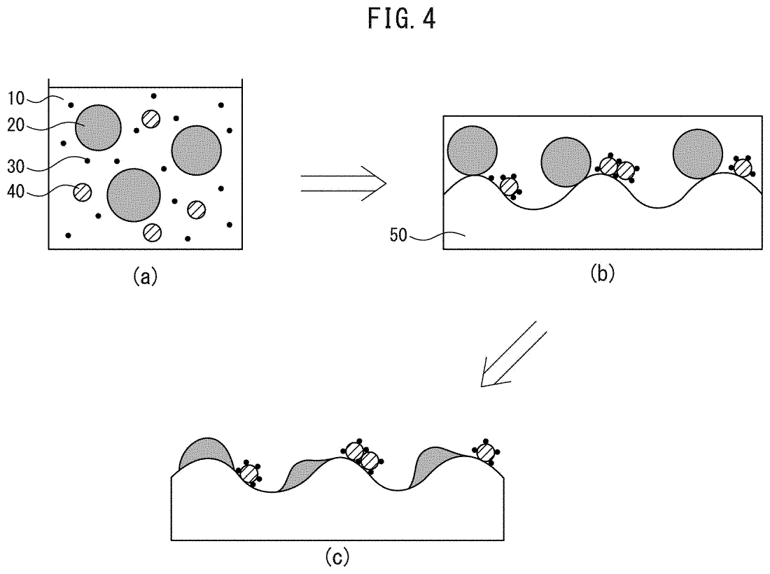

In the case of an emulsified cosmetic containing hydrophobically treated microparticles 30 and pigment grade hydrophobically treated particles 40 in an aqueous phase as shown in FIG. 4, since the hydrophobically treated particles are hardly dispersed in the aqueous phase in the first place, it is considered that both particles tend to aggregate in the aqueous phase. As a result, it is considered that, since both of the aggregated particles are disposed on the skin surface, defects such as color unevenness or tone-up unevenness tend to occur, and each function exhibited by both particles cannot be sufficiently exerted.

Further, since the hydrophobically treated microparticles have a smaller particle diameter than the pigment grade hydrophobically treated particles, the specific surface area becomes large. As a result, when it is attempted to disperse the hydrophobically treated microparticles in the aqueous phase, it is necessary to blend a larger amount of the nonionic surfactant than in the case where only the pigment grade hydrophobically treated particles are dispersed in the aqueous phase, so that a sticky feeling associated with the nonionic surfactant tends to occur.

### Oil-in-Water Type Emulsified Cosmetics

### Dispersion Medium

The dispersion medium in the oil-in-water type emulsified cosmetic of the present disclosure contains water, a pigment grade hydrophobically treated particle, a nonionic surfactant, and a polyhydric alcohol.

### (Water)

The amount of water to be blended is not particularly limited, but may be, for example, from the viewpoint of emulsification stability and the like, 30% by mass or more, 40% by mass or more, 50% by mass or more, 60% by mass or more, 70% by mass or more, or 80% by mass or more, based on the total amount of the cosmetic, and may be 90% by mass or less, 80% by mass or less, 70% by mass or less, or 60% by mass or less.

The water which can be used in the oil-in-water type emulsified cosmetic of the present disclosure is not particularly limited, but water used in cosmetics, quasi-drugs, and the like can be used. For example, ion-exchanged water, distilled water, ultrapure water, tap water, or the like can be used.

### (Pigment Grade Hydrophobically Treated Particle)

The amount of the pigment grade hydrophobically treated particles to be blended is not particularly limited, and can be appropriately selected based on a desired effect (e.g., a tone-up effect) depending on the application, and for example, it can be 0.5% by mass or more, 1.0% by mass or more, or 1.5% by mass or more, based on the total amount of the cosmetic, and can be 15% by mass or less, 12% by mass or less, 10% by mass or less, 8.0% by mass or less, 6.0% by mass or less, or 5.0% by mass or less.

The cosmetic of the present disclosure contains pigment grade hydrophobically treated particles in a dispersion medium. The pigment grade hydrophobically treated particles and the hydrophobically treated microparticles described later can be distinguished by their size. In other words, the pigment grade hydrophobically treated particles may refer to hydrophobically treated particles having an average particle diameter of 300 nm or more, and the hydrophobically treated microparticles may refer to hydrophobically treated particles having an average particle diameter smaller than that of the pigment grade hydrophobically treated particles, for example, an average particle diameter of 200 nm or less. Here, the average particle diameter of the pigment grade hydrophobically treated particles and the hydrophobically treated microparticles described later in the present disclosure may be a size of the primary particles or the aggregated secondary particles, and can be calculated by a static light scattering method.

The pigment grade hydrophobically treated particles having an average particle diameter of 300 nm or more can exhibit a tone-up effect or the like of lightening the color of the skin. The average particle diameter of the pigment grade hydrophobically treated particles can be appropriately selected so as to obtain a desired tone-up effect, and can be, for example, 300 nm or more, 350 nm or more, or 400 nm or more. Although there is no particular limitation on the upper limit value of the average particle diameter of the pigment grade hydrophobically treated particles, for example, it may be 800 nm or less, 700 nm or less, or 600 nm or less.

There is no particular limitation on the hydrophobization treatment of the pigment grade hydrophobically treated particles, examples thereof include optional treatment for hydrophobizing the surface of such particles by modifying with an organic compound, for example, silicone-based treatment or silane-based treatment with methylhydrogenpolysiloxane, dimethylpolysiloxane, alkylsilane, or the like; fluorine-based treatment with a perfluoroalkyl phosphate ester, perfluoroalcohol, or the like; titanate-based treatment with an alkyl titanate or the like; amino acid treatment with a N-acylglutamic acid or the like, and other treatment include lecithin treatment; metal soap treatment; fatty acid treatment; alkyl phosphate ester treatment, and the like. These may be used alone or in combination of two or more thereof.

From the viewpoint of resistance to release from particles and the like, the hydrophobization treatment is preferably carried out with a reactive hydrophobization treatment agent such as a silicone, a silane-based treatment agent, a titanate-based treatment agent or the like.

Examples of the silicone as such a hydrophobization treatment agent may include a known silicone having a hydrogen-silicon bond such as a methyl hydrogenpolysiloxane (dimethicone/methicone) copolymer, and the like. Further, triethoxysilylethylpolydimethylsiloxyethyldimethicone, triethoxysilylethylpolydimethylsiloxyethylhexyldimethicone, and the like, which have an alkoxy group-silicon bond as a reactive group, can also be included. In addition, dimethylpolysiloxane and the like can also be used.

Examples of the silane-based treatment agent may include a silylating agent having an organic group introduced therein and a silane coupling agent, and examples thereof may include triethoxycaprylylsilane.

Examples of the titanate-based treating agent may include a titanium coupling agent such as an alkyl titanate, a titanate of a pyrophosphate type, a titanate of a phosphite type, a titanate of an amino acid type, and the like.

From the viewpoint of affinity to the skin, cost, and the like, the hydrophobization treatment agent is preferably applied to the particles at a ratio of 2% by mass or more, 3% by mass or more, or 5% by mass or more, 12% by mass or less, 10% by mass or less, or 8% by mass or less, based on the total amount of the pigment grade hydrophobically treated particles after treatment.

The type of particles constituting the pigment grade hydrophobically treated particles is not particularly limited, and can be appropriately selected so as to obtain a desired tone-up effect or the like. Examples of the pigment grade hydrophobically treated particles may include inorganic particles, specifically, inorganic oxide particles, for example, white based inorganic oxide particles (sometimes referred to as “inorganic white based pigment”) such as a titanium oxide particle, a zinc oxide particle, and a cerium oxide particle, and the like. In addition, in general, inorganic particles and the like classified as a pearl agent (glittering pigment) or a coloring material can also be used as the pigment grade hydrophobically treated particles of the present disclosure. Organic particles may also be used as the pigment grade hydrophobically treated particles of the present disclosure. These may be used alone or in combination of two or more thereof.

In the present disclosure, a “pearl agent” is intended to be a particle which does not include a coloring material and exhibits glitter. A pearl agent typically has a flattened form such as flaky or scaly. In addition, in the present disclosure, a “coloring material” is intended to be a material which exhibits a color other than white, does not include a pearl agent, and does not exhibit a glittering property capable of developing color of a cosmetic. In view of the tone-up effect, when a pearl agent is used, it is preferable to use it in combination with inorganic oxide particles such as titanium oxide particles described above, and when a coloring material is used, it is preferable to use it in combination with inorganic oxide particles such as titanium oxide particles described above and/or a pearl agent.

Examples of the pearl agent may include mica titanium (titanated mica), iron oxide-coated mica titanium, carmine-coated mica titanium, carmine and iron blue-coated mica titanium, iron oxide and carmine-treated mica titanium, iron blue-treated mica titanium, iron oxide and iron blue treated mica titanium, chromium oxide-treated mica titanium, black titanium oxide-treated mica titanium, acrylic resin-coated aluminum powder, silica-coated aluminum powder, titanium oxide-coated mica, titanium oxide-coated bismuth oxychloride, titanium oxide-coated talc, colored titanium oxide-coated mica, titanium oxide-coated synthetic mica, titanium oxide-coated silica, titanium oxide-coated alumina, titanium oxide-coated glass powder, polyethylene terephthalate and polymethyl methacrylate laminated film powder, bismuth oxychloride, fish scale foil, iron oxide and titanium oxide-coated mica, such as red oxide and titanium oxide-coated mica, which is mica coated with iron oxide and titanium oxide, powdered hollow titanium oxide with silica between mica and titanium oxide coating layer, or the like. These typically exhibit a white or other color.

As the pearl agent, a colorless pearl agent may also be used. As such a pearl agent, a known transparent pearl agent (transparent glittering pigment) can be used. Examples thereof may include a pearl agent obtained by forming a coating composed of a high refractive index material such as titanium dioxide on the surface of a glass particle as a substrate.

As the coloring material, for example, an inorganic pigment can be used.

Examples of the inorganic pigment may include an inorganic red pigment (e.g., iron oxide (red oxide), iron titanate, etc.); an inorganic brown pigment (e.g., γ-iron oxide, etc.); an inorganic yellow pigment (e.g., yellow iron oxide, ocher, etc.); an inorganic black pigment (e.g., black iron oxide, low-order titanium oxide, etc.); an inorganic purple pigment (e.g., manganese violet, cobalt violet, etc.); an inorganic green pigment (e.g., chromium oxide, chromium hydroxide, cobalt titanate, etc.); an inorganic blue pigment (e.g., ultramarine blue, iron blue, etc.); a metal powder (e.g., aluminum, gold, silver, copper, etc.).

### (Nonionic Surfactant)

The content of the nonionic surfactant in the oil-in-water type emulsified cosmetic of the present disclosure is not particularly limited, and may be, for example, 0.03% by mass or more, 0.05% by mass or more, 0.07% by mass or more, or 0.1% by mass or more, based on the total amount of the cosmetic, and may be 3.0% by mass or less, 2.5% by mass or less, 2.0% by mass or less, 1.5% by mass or less, 1.0% by mass or less, or 0.5% by mass or less. From its performance, it is considered that the nonionic surfactant is present in the dispersion medium, on the surface of the pigment grade hydrophobically treated particles, and at the interface of the oil droplet described later. In the aqueous phase, it is considered that the nonionic surfactant contributes to the dispersion stability of the pigment grade hydrophobically treated particles.

There is no particular limitation on the nonionic surfactant, and for example, a nonionic surfactant having an HLB of 10.0 or more, 12.0 or more, or 14.0 or more, or 16.0 or less, 15.5 or less, or 15.0 or less can be used. A nonionic surfactant having such an HLB can further improve the emulsification stability of oil droplets and the dispersion stability of pigment grade hydrophobically treated particles in an aqueous phase. Among them, a nonionic surfactant having an HLB of 14.0 to 16.0 is preferred. In addition, in some embodiments, the cosmetic of the present disclosure may be used in combination with a nonionic surfactant having an HLB of less than 10.0, for example, an HLB of 9.0 or less or 8.5 or less, 3.0 or more, 3.5 or more, or 4.0 or more. Here, HLB is a value which generally indicates affinity of a surfactant to water and oil, and is a parameter known as a hydrophilic-lipophilic balance, and can be easily determined by a known calculation method such as a griffin method.

The nonionic surfactant is not particularly limited, and examples thereof may include polyoxyalkylene alkyl ethers, polyalkylene glycol fatty acid esters, POE hardened castor oil derivatives, POE alkyl ethers, POE⋅POP alkyl ethers, PEG fatty acid esters, polyglycerin fatty acid esters, POE glycerin fatty acid esters, PEG glyceryl isostearate, and silicone surfactants. In addition, nonionic surfactants such as polyglyceryl-2 diisostearate and sorbitan sesquiisostearate may also be used. The nonionic surfactants may be used alone or in combination of two or more thereof.

Examples of the polyoxyalkylene alkyl ether may include polyoxyethylene behenyl ether and polyoxyethylene stearyl ether.

Examples of the polyalkylene glycol fatty acid ester may include polyethylene glycol monostearate and polyethylene glycol monooleate.

Examples of the POE hardened castor oil derivative (PEG hydrogenated castor oil) may include POE (20 to 100) hardened castor oil derivatives. Specific examples thereof may include POE (20) hardened castor oil derivative, POE (40) hardened castor oil derivative, POE (60) hardened castor oil derivative, and POE (100) hardened castor oil derivative.

Examples of the POE alkyl ethers may include POE (2) lauryl ether, POE (4.2) lauryl ether, POE (9) lauryl ether, POE (5.5) cetyl ether, POE (7) cetyl ether, POE (10) cetyl ether, POE (15) cetyl ether, POE (20) cetyl ether, POE (23) cetyl ether, POE(4) stearyl ether, POE (20) stearyl ether, POE (7) oleyl ether, POE (10) oleyl ether, POE (15) oleyl ether, POE (20) oleyl ether, POE (50) oleyl ether, POE (10) behenyl ether, POE (20) behenyl ether, POE (30) behenyl ether, POE(2) (C12 to 15) alkyl ether, POE(4) (C12 to 15) alkyl ether, POE (10) (C12 to 15) alkyl ether, POE (5) secondary alkyl ether, POE (7) secondary alkyl ether, POE (9) alkyl ether, POE (12) alkyl ether, and the like.

Examples of the POE⋅POP alkyl ethers may include POE (1) polyoxypropylene (POP) (4) cetyl ether, POE (10) POP (4) cetyl ether, POE (20) POP (8) cetyl ether, POE (20) POP (6) decyl tetradecyl ether, POE (30) POP (6) decyl tetradecyl ether, and the like.

Examples of the PEG fatty acid esters may include polyethylene glycol (hereinafter abbreviated as PEG) (10) monolaurate, PEG (10) monostearate, PEG (25) monostearate, PEG (40) monostearate, PEG (45) monostearate, PEG (55) monostearate, PEG (100) monostearate, PEG (10) monooleate, PEG distearate, PEG diisostearate, and the like.

Examples of the polyglycerin fatty acid esters may include hexaglyceryl monolaurate, hexaglyceryl monomyristate, hexaglyceryl monostearate, hexaglyceryl monooleate, decaglyceryl monolaurate, decaglyceryl monomyristate, decaglyceryl monostearate, decaglyceryl monoisostearate, decaglyceryl monooleate, decaglyceryl distearate, decaglyceryl diisostearate, and the like.

Examples of the POE glycerin fatty acid esters may include polyoxyethylene (POE) (5) glyceryl monostearate, POE (15) glyceryl monostearate, POE (5) glyceryl monooleate, POE (15) glyceryl monooleate, and the like.

Examples of the PEG glyceryl isostearate may include PEG (8) glyceryl isostearate, PEG (10) glyceryl isostearate, PEG (15) glyceryl isostearate, PEG (20) glyceryl isostearate, PEG (25) glyceryl isostearate, PEG glyceryl (30) isostearate, PEG (40) glyceryl isostearate, PEG (50) glyceryl isostearate, PEG (60) glyceryl isostearate, and the like.

As silicone surfactants, for example, polyether-modified silicone can be employed, and examples thereof may include PEG (3) dimethicone, PEG (7) dimethicone, PEG (9) dimethicone, PEG (10) dimethicone, PEG (12) dimethicone, PEG (9) methyl ether dimethicone, PEG (10) methyl ether dimethicone, PEG (11) methyl ether dimethicone, PEG (32) methyl ether dimethicone, and PEG (9) polydimethylsiloxyethyl dimethicone.

Among them, from the viewpoint of emulsification stability of oil droplets, dispersibility of pigment grade hydrophobically treated particles, and the like, polyether-modified silicone is preferred, and PEG (11) methyl ether dimethicone is more preferred.

From the viewpoint of emulsification stability of oil droplets, dispersibility of pigment grade hydrophobically treated particles, usability such as sticky feeling, and the like, the mass ratio of the pigment grade hydrophobically treated particle to the polyether-modified silicone having an HLB of 10.0 to 16.0 is preferably, for example, 11 or more, 12 or more, 13 or more, or 14 or more, and is preferably 30 or less, 28 or less, or 25 or less.

### (Polyhydric Alcohol)

There is no particular limitation on the content of the polyhydric alcohol in the oil-in-water type emulsified cosmetic of the present disclosure, and from the viewpoint of tone-up property, dispersion stability of the pigment grade hydrophobically treated particles, and the like, for example, it can be 1.5% by mass or more, 2.0% by mass or more, 2.5% by mass or more, or 3.0% by mass or more, preferably 3.5% by mass or more, more preferably 4.0% by mass or more, and still more preferably 4.5% by mass or more, based on the total amount of the cosmetic. The upper limit for the content of the polyhydric alcohol is not particularly limited, but it can be 10% by mass or less, 8.0% by mass or less, 6.0% by mass or less, or 5.0% by mass or less. The polyhydric alcohol is present in the dispersion medium and on the surface of the pigment grade hydrophobically treated particles, and is considered to contribute to the dispersion stability of the pigment grade hydrophobically treated particles, but may be contained in an oil droplet described later.

Examples of the polyhydric alcohol may include ethylene glycol, propylene glycol, 1,3-butylene glycol, dipropylene glycol, polyethylene glycol, polypropylene glycol, and polybutylene glycol. Among them, 1,3-butylene glycol is preferred from the viewpoint of dispersibility and the like of the pigment grade hydrophobically treated particles. These may be used alone or in combination of two or more thereof. Note that the “polyhydric alcohol” in the present disclosure does not include glycerin.

### Oil Droplet

An oil droplet as an oil phase or a dispersed phase in an oil-in-water type emulsified cosmetic contains an oil content, a nonionic surfactant, and a hydrophobically treated microparticle having an average particle diameter of 200 nm or less. Here, as the nonionic surfactant, the above-described nonionic surfactant can be similarly employed. Further, the oil droplet may contain the above-described polyhydric alcohol.

### (Oil Content)

The content of the oil content in the oil-in-water type emulsified cosmetic of the present disclosure is not particularly limited, and may be, for example, 10% by mass or more, 12% by mass or more, 15% by mass or more, 17% by mass or more, 20% by mass or more, 22% by mass or more, or 25% by mass or more, based on the total amount of the cosmetic, and may be 50% by mass or less, 40% by mass or less, 30% by mass or less, or 25% by mass or less.

Since a cosmetic having a configuration in which an oil content is highly contained in an amount of, for example, 20% by mass or more can increase, for example, a content ratio of an ultraviolet absorber, and a hydrophobically treated microparticle as an ultraviolet scattering agent, an ultraviolet protective effect (SPF) can be further improved.

The oil-in-water type emulsified cosmetic of the present disclosure contains a volatile oil as an oil content. Here, “volatile” is intended to be one in which the volatile content when left at 105° C. for 3 hours under atmospheric pressure exhibits more than 5%. From the viewpoint of uniformly applying the pigment grade hydrophobically treated particles and the hydrophobically treated microparticles to the skin, the volatile content serving as a guide for volatility is preferably 10% or more, 20% or more, 40% or more, 50% or more, 60% or more, 80% or more, or 100%. Alternatively, a boiling point under 1 atm (101.325 kPa) can be used as a guide for volatility. This boiling point is preferably 250° C. or less, 240° C. or less, or 230° C. or less from the viewpoint of uniformly applying the pigment grade hydrophobically treated particles and the hydrophobically treated microparticles to the skin, and is preferably 80° C. or more, 100° C. or more, 120° C. or more, 150° C. or more, or 160° C. or more. In addition, in the present disclosure, “nonvolatile” is intended to be one in which the volatile content when left at 105° C. for 3 hours exhibits 5% or less.

There is no particular limitation on the volatile oil, and examples thereof may include volatile silicone oil, and volatile hydrocarbon oil. The volatile oils may be used alone or in combination of two or more thereof.

Examples of the volatile silicone oil may include volatile acyclic silicone oil, and volatile cyclic silicone oil. Among them, volatile acyclic silicone oil is preferred.

As volatile acyclic silicone oil, for example, volatile linear silicone oil, and volatile branched silicone oil can be used. Among them, volatile linear silicone oil is preferred.

Examples of the volatile linear silicone oil include low molecular weight linear dimethylpolysiloxane such as dimethylpolysiloxane (sometimes referred to as “dimethicone”) having a viscosity of 0.65 cSt, dimethylpolysiloxane having a viscosity of 1 cSt, dimethylpolysiloxane having a viscosity of 1.5 cSt, and dimethylpolysiloxane having a viscosity of 2 cSt. Among them, dimethylpolysiloxane having a viscosity of 1 cSt and dimethylpolysiloxane having a viscosity of 1.5 cSt are preferred from the viewpoint of uniformly applying the pigment grade hydrophobically treated particles and the hydrophobically treated microparticles to the skin. Here, these viscosities are intended to be the kinematic viscosity under 25° C. atmosphere.

Examples of the volatile branched silicone oil include low molecular weight branched siloxane such as methyltrimethicone, tris (trimethylsilyl) methylsilane, and tetrakis (trimethylsilyl) silane.

Examples of the volatile cyclic silicone oil include octamethylcyclotetrasiloxane, decamethylcyclopentasiloxane, and dodecamethylcyclohexasiloxane.

Examples of the volatile hydrocarbon oil may include heptane, isododecane, isohexadecane, and isodecane. Among them, isododecane is preferred from the viewpoint of uniformly applying the pigment grade hydrophobically treated particles and the hydrophobically treated microparticles to the skin.

There is no particular limitation on the content of the volatile oil in the oil-in-water type emulsified cosmetic of the present disclosure, and for example, from the viewpoint of uniformly applying the pigment grade hydrophobically treated particles and the hydrophobically treated microparticles to the skin, the content is preferably 1% by mass or more, 2% by mass or more, 3% by mass or more, 4% by mass or more, 5% by mass or more, 6% by mass or more, 7% by mass or more, 8% by mass or more, 9% by mass or more, or 10% by mass or more, based on the total amount of the cosmetic, and is preferably 30% by mass or less, 25% by mass or less, or 20% by mass or less. Alternatively, the content of the volatile oil is preferably 10% by mass or more, 15% by mass or more, 17% by mass or more, 20% by mass or more, 25% by mass or more, 30% by mass or more, 35% by mass or more, 40% by mass or more, 45% by mass or more, or 50% by mass or more, based on the total amount of the oil content, and is preferably about 100% by mass or less, less than about 100% by mass, about 90% by mass or less, about 80% by mass or less, about 70% by mass or less, or about 60% by mass or less.

The oil-in-water type emulsified cosmetic of the present disclosure may be blended with other oil contents other than volatile oils, for example, nonvolatile oils. There is no particular limitation on such oil contents, and examples thereof may include an oil content commonly used in cosmetics, for example, a liquid oil and fat, a solid oil and fat, waxes, a hydrocarbon oil other than the above, a silicone oil other than the above, a polar oil, and the like. When other oil contents (e.g., nonvolatile oils) are used in combination with volatile oils, particles can be suitably immobilized on the skin because such other oil contents can function as a binder between the particles and the skin after the volatile oils have volatilized. Other oil contents may be used alone or in combination of two or more thereof. Here, among the ultraviolet absorbers, some ultraviolet absorbers act as an oil content, especially a polar oil. Such an ultraviolet absorber can also be regarded as an oil content.

Examples of the liquid oil and fat include avocado oil, camellia oil, turtle oil, macadamia nut oil, corn oil, mink oil, olive oil, rape seed oil, egg yolk oil, sesame oil, persic oil, wheat germ oil, camellia sinensis leaf oil, castor oil, linseed oil, safflower oil, cottonseed oil, perilla oil, soybean oil, peanut oil, camellia sinensis seed oil, torreya nucifera oil, oryza sativa bran oil, China tung oil, Japanese tung oil, jojoba oil, germ oil, triglycerin, and the like.

Examples of the solid oil and fat include cocoa butter, coconut oil, horse fat, hardened coconut oil, palm oil, beef tallow, mutton tallow, hardened beef tallow, palm kernel oil, lard, beef bone fat, Japan tallow kernel oil, hardened oil, neatsfoot fat, Japan tallow, hardened castor oil, and the like.

Examples of the waxes include yellow bees wax, candelilla wax, cotton wax, carnauba wax, bayberry wax, tree wax, spermaceti wax, montan wax, bran wax, lanolin, kapok wax, acetic acid lanolin, liquid lanolin, sugarcane wax, isopropyl lanolate, hexyl laurate, reduced lanolin, jojoba wax, hard lanolin, shellac wax, POE lanolin alcohol ether, POE lanolin alcohol acetate, POE cholesterol ether, polyethylene glycol laurate, POE hydrogenated lanolin alcohol ether, and the like.

Examples of the hydrocarbon oil include liquid paraffin, ozokerite, squalane, pristane, paraffin, ceresin, squalene, petrolatum, microcrystalline wax, and olefin oligomer, and the like.

Examples of the silicone oil include chain silicones such as dimethylpolysiloxane (dimethicone) having a viscosity of 6 cSt or more, methylphenylpolysiloxane (diphenylsiloxyphenyltrimethicone), and methylhydrogenpolysiloxane.

As the polar oil, for example, a polar oil having an IOB of 0.10 or more can be used. Examples of such a polar oil include isopropyl myristate (IOB value=0.18), octyl palmitate (IOB value=0.13), isopropyl palmitate (IOB value=0.16), butyl stearate (IOB value=0.14), hexyl laurate (IOB value=0.17), myristyl myristate (IOB value=0.11), decyl oleate (IOB value=0.11), isononyl isononate (IOB value=0.20), isotridecyl isononate (IOB value=0.15), cetyl ethylhexanoate (IOB value=0.13), pentaerythrityl tetraethylhexanoate (IOB value=0.35), diethylhexyl succinate (IOB value=0.32), dioctyl succinate (IOB value=0.36), glycol distearate (IOB value=0.16), glyceryl diisostearate (IOB value=0.29), neopentyl glycol dicapurinate (IOB value=0.25), diisostearyl malate (IOB value=0.28), trimethylolpropane triisostearate (IOB value=0.16), glyceryl tri2-ethylhexanoate (triethylhexanoin) (IOB value=0.35), trimethylolpropane trioctanoate (IOB value=0.33), trimethylolpropane triisostearate (IOB value=0.16), diisobutyl adipate (IOB value=0.46), N-lauroyl-L-glutamic acid-2-octyldodecyl ester (IOB value=0.29), 2-hexyldecyl adipate (IOB value=0.16), diisopropyl sebacate (IOB value=0.40), ethylhexyl methoxycinnamate (IOB value=0.28), 2-ethylhexyl palmitate (IOB value=0.13), 2-ethylhexyl ethylhexanoate (IOB value=0.2), triisostearin (IOB value=0.16), PPG-3 dipivalinate (IOB value=0.52), tri (caprylic acid/capric acid) glyceryl (IOB value=0.33), and the like.

Examples of ultraviolet absorbers which can be considered as an oil content may include ultraviolet absorbers having an IOB of 0.10 or more, specifically, organic ultraviolet absorbers such as ethylhexyl methoxycinnamate, octocrylene, polysilicone-15, t-butylmethoxydibenzoylmethane, ethylhexyltriazone, bisethylhexyloxyphenol methoxyphenyltriazine, hexyl diethylaminohydroxybenzoylbenzoate, oxybenzone-3, methylenebisbenzotriazolyltetramethylbutylphenol, homosalate, and ethylhexyl salicylate. These ultraviolet absorbers may be used alone or in combination of two or more thereof.

The IOB value of the polar oil and the ultraviolet absorber may be, for example, 0.11 or more, 0.12 or more, or 0.13 or more, and may be 0.50 or less, 0.45 or less, or 0.40 or less. Here, the IOB value is an abbreviation of Inorganic/Orgnic Balance (inorganic/organic ratio), and is a value representing a ratio of an inorganic value to an organic value, and is an indicator indicating a degree of polarity of an organic compound. The IOB value is specifically expressed as the IOB value=inorganic value/organic value. For each of the “inorganic value” and the “organic value”, an “inorganic value” and an “organic value” corresponding to various atoms or functional groups are set (for example, an “organic value” is 20 for one carbon atom in a molecule and an “inorganic value” is 100 for one hydroxyl group), and the IOB value of the organic compound can be calculated by integrating the “inorganic value” and the “organic value” of all atoms and functional groups in an organic compound (see, for example, written by Yoshio Koda, “Organic Conceptual Chart—Basics and Applications—”, p.11 to 17, Sankyo Publishing, 1984).

In the cosmetic of the present disclosure, at least one selected from the above-mentioned polar oils and ultraviolet absorbers can be relatively highly blended as an oil content. These oil contents may contain 10% by mass or more, 15% by mass or more, 20% by mass or more, 25% by mass or more, 30% by mass or more, 35% by mass or more, 40% by mass or more, or 45% by mass or more, based on the total oil content. There is no particular limitation on the upper limit value of these oil contents, and for example, it may be 90% by mass or less, 85% by mass or less, 80% by mass or less, 75% by mass or less, 70% by mass or less, 65% by mass or less, 60% by mass or less, 55% by mass or less, or 50% by mass or less.

### (Hydrophobically Treated Microparticle)

The amount of the hydrophobically treated microparticles to be blended is not particularly limited, and can be appropriately selected based on a desired effect (e.g., ultraviolet scattering effect) depending on the application, and for example, it may be 0.5% by mass or more, 1.0% by mass or more, 1.5% by mass or more, 2.0% by mass or more, 2.5% by mass or more, 3.0% by mass or more, 3.5% by mass or more, 4.0% by mass or more, 4.5% by mass or more, or 5.0% by mass or more, based on the total amount of the cosmetic, and 20% by mass or less, 17% by mass or less, 15% by mass or less, 13% by mass or less, 10% by mass or less, 8.0% by mass or less, 6.0% by mass or less, or 5.0% by mass or less.

The hydrophobically treated microparticles having an average particle diameter of 200 nm or less can exert, for example, an ultraviolet scattering effect or the like. The average particle diameter of the hydrophobically treated microparticles can be appropriately selected based on a desired effect (e.g., ultraviolet scattering effect) depending on the application, for example, it may be 200 nm or less, 180 nm or less, 150 nm or less, 120 nm or less, 100 nm or less, or 80 nm or less. There is no particular limitation on the lower limit value of the average particle diameter of the hydrophobically treated microparticles, for example, it may be 10 nm or more, 20 nm or more, 30 nm or more, 40 nm or more, 50 nm or more, 60 nm or more, or 70 nm or more.

There is no particular limitation on the hydrophobization treatment of the hydrophobically treated microparticles, and for example, the same treatment as that of the hydrophobization treatment in the pigment grade hydrophobically treated particles described above can be performed.

The type of particles constituting the hydrophobically treated microparticles is not particularly limited, and may be appropriately selected based on a desired effect (e.g., ultraviolet scattering effect) depending on the application, and examples thereof may include inorganic particles, specifically, titanium oxide, zinc oxide, barium sulfate, iron oxide, talc, mica, sericite, kaolin, mica titanium, iron blue, chromium oxide, chromium hydroxide, silica, cerium oxide, and the like. These may be used alone or in combination of two or more thereof. When the hydrophobically treated microparticles are used as an ultraviolet scattering agent, it is preferable to use particles having a refractive index of 1.5 or more, for example, zinc oxide particles or titanium oxide particles, from the viewpoint of optical characteristics and the like.

### Optional Ingredients

In the oil-in-water type emulsified cosmetic of the present disclosure, various ingredients can be appropriately blended within a range that does not affect the effect of the present disclosure. Examples of various ingredients may include additive ingredients which can be usually blended into a cosmetic. Examples thereof may include an anionic surfactant, a cationic surfactant, an amphoteric surfactant, a moisturizing agent, a thickener, a water-soluble polymer, an oil-soluble polymer, a film-forming agent such as siliconized polysaccharide, a higher fatty acid such as isostearic acid, a metal ion sequestering agent, a lower alcohol such as ethanol, a higher alcohol such as stearyl alcohol, various extracts, a sugar, an amino acid, an organic amine, a polymeric emulsion, a chelating agent, other ultraviolet absorbers other than the above-mentioned ultraviolet absorbers, a pH adjuster, a skin nutrition agent, a vitamin, a pharmaceutical product, a quasi-drug, a water-soluble drug applicable to cosmetics and the like, a buffering agent, a discoloration inhibitor, a preservative, a dispersant, a propellant, an organic powder, other pigments (e.g., organic pigments) other than those that can be used in the above pearl agents and coloring materials, a dye, a colorant, a perfume, and the like.

### Cosmetic Viscosity

In some embodiments, the viscosity of the oil-in-water type emulsified cosmetic of the present disclosure, in which the viscosity is measured using the conditions and apparatus described in the examples described later, may be 20,000 mPa·s or less, 15,000 mPa·s or less, or 10,000 mPa·s or less, immediately after preparation of the cosmetic, and may be 500 mPa·s or more, 1,000 mPa·s or more, 1,500 mPa·s or more, 2,000 mPa·s or more, or 2,500 mPa·s or more. Such viscosity of the cosmetic immediately after preparation can be referred to as “initial viscosity”.

In some embodiments, the viscosity of the oil-in-water type emulsified cosmetic of the present disclosure, in which the viscosity is measured using the conditions and apparatus described in the examples described later, may be 20,000 mPa·s or less, 15,000 mPa·s or less, or 10,000 mPa·s or less, after one day of preparation of the cosmetic, and may be 500 mPa·s or more, 1,000 mPa·s or more, 1,500 mPa·s or more, 2,000 mPa·s or more, or 2,500 mPa·s or more. Such viscosity of the cosmetic after one day of preparation can be referred to as “storage viscosity”.

### Method for Preparing Oil-in-Water Type Emulsified Cosmetic

The method for preparing the oil-in-water type emulsified cosmetic of the present disclosure is not particularly limited, for example, can be prepared by a known method such as a dispersion method or an aggregation method.

The dispersion method is a method in which a mass of a dispersed phase is finely divided by mechanical force. Specifically, it is a method of emulsifying using a crushing force of an emulsifier, and examples of such a method may include a high pressure emulsification method in which a high shear force is applied using a high pressure homogenizer, and the like.

The agglomeration method is a method for preparing colloids which utilizes interfacial chemical characteristics, and is a method in which a uniformly dissolved state is made into a supersaturated state by a certain means, whereby a dispersed phase appears. As specific examples of such a method, an HLB temperature emulsification method, a phase inversion emulsification method, a non-aqueous emulsification method, a D-phase emulsification method, a liquid crystal emulsification method, and the like are known.

### Dosage Form for Oil-in-Water Type Emulsified Cosmetic

There is no particular limitation on the dosage form of the oil-in-water type emulsified cosmetic of the present disclosure, and examples thereof include liquid, emulsion, cream, gel, spray, mousse, and the like. Here, “spray” in the present disclosure may encomPa·ss a mist type spray, an aerosol type spray, and the like.

### Application of Oil-in-Water Type Emulsified Cosmetic

The oil-in-water type emulsified cosmetic of the present disclosure can apply hydrophobically treated particles and optionally ultraviolet absorbers uniformly to the skin surface to exert a good tone-up effect and the like. Thus, the cosmetic of the present disclosure that can exhibit such performance can be used, for example, as a cosmetic applied by spreading on the skin or the like. Here, the cosmetic applied to the skin may also include what is called an external preparation for skin.

The product form of the cosmetic of the present disclosure is not particularly limited, and examples thereof may include facial cosmetics such as a skin lotion, a beauty liquid, an emulsion, and a pack; makeup cosmetics such as a foundation, a lipstick, and an eye shadow; sunscreen cosmetics (sunscreen agents); body cosmetics; skin cleansers such as a makeup-remover and a body shampoo; hair cosmetics such as a hair liquid, a hair tonic, a hair conditioner, a shampoo, a rinse, and a hair growth agent; an ointments, and the like.

### EXAMPLES

Hereinafter, the oil-in-water type emulsified cosmetic of the present disclosure will be described in more detail with reference to Examples, but the cosmetic of the present disclosure is not limited thereto. Note that, hereinafter, unless otherwise specified, the blending amount is indicated % by mass.

### Examples 1 to 13 and Comparative Examples 1 to 6

The following evaluations were performed on the oil-in-water type emulsified cosmetics obtained by the formulations shown in Tables 1 to 3 and the manufacturing method shown below, and the results thereof are shown in Tables 1 to 3.

### Evaluation Method

### (Evaluation of Tone-Up)

The prepared cosmetic was applied to an arm to dry moisture, and the cosmetic-applied surface after standing for 5 minutes in a state in which the arm was hanging in the ground direction was visually observed, and the state of the tone-up was evaluated on the basis of the following criteria. Here, up to A and B evaluation can be regarded as acceptable and C evaluation as unacceptable. Further, in addition to the tone-up effect, this tone-up test can indirectly evaluate whether or not the pigment grade hydrophobically treated particles and the hydrophobically treated microparticles are uniformly applied to the skin surface. In other words, it can be said that particles are uniformly applied to the skin surface in the order of C, B, and A.

A: There was no unevenness in brightness, and an excellent tone-up effect was exhibited.
B: A slight unevenness in brightness occurred, but a good tone-up effect was obtained.
C: There was apparent unevenness in brightness, and a good tone-up effect was not obtained.

### (Evaluation of Particle Dispersibility)

The prepared cosmetic was poured into a 50 mL transparent sample tube (diameter: 3 cm), and the dispersion state of the pigment grade hydrophobically treated particles and the hydrophobically treated microparticles after storage at 25° C. for 7 days was visually observed and evaluated on the basis of the following criteria.

A: No precipitates of the pigment grade hydrophobically treated particles and the hydrophobically treated microparticles were confirmed.
B: Precipitates of the pigment grade hydrophobically treated particles and the hydrophobically treated microparticles were slightly confirmed.
C: Precipitates of the pigment grade hydrophobically treated particles and the hydrophobically treated microparticles were clearly confirmed.

### (Evaluation of Rolling Stability)

The prepared cosmetic was poured into a 50 mL transparent sample tube (diameter: 3 cm), and the sample tube was rotated under an atmosphere of 25° C. at a speed of 45 rpm for 4 hours, and the aggregation state of the pigment grade hydrophobically treated particles and the hydrophobically treated microparticles was visually observed and evaluated on the basis of the following criteria.

A: No color stripe pattern associated with the agglomerates of the pigment grade hydrophobically treated particles and the hydrophobically treated microparticles was observed.
B: Very slight color stripe patterns associated with the agglomerates of the pigment grade hydrophobically treated particles and the hydrophobically treated particles were observed.
C: Slight color stripe patterns associated with the agglomerates of the pigment grade hydrophobically treated particles and the hydrophobically treated particles were observed.
D: Color stripe patterns associated with the agglomerates of the pigment grade hydrophobically treated particles and the hydrophobically treated particles were clearly observed.

### (Evaluation of Usability)

The prepared cosmetic was applied to the skin, and the smoothness, sticky feeling, and powder squeakiness feeling, at the time of application, during or after application were evaluated by five expert panels according to the following evaluation criteria.

A: Five respondents answered that they felt smooth feeling of use without feeling sticky and powder squeakiness.
B: Three to four respondents answered that they felt smooth feeling of use without feeling sticky and powder squeakiness.
C: Zero to two respondents answered that they felt smooth feeling of use without feeling sticky and powder squeakiness.

### (Evaluation of Viscosity)

The viscosity of the cosmetic was evaluated using a B-type viscometer (TVB-type viscometer TVB-10, manufactured by Toki Sangyo Co., Ltd.) under conditions of rotor number 3, 30° C., and 12 rpm. Note that measurement of the viscosity was performed on the viscosity after one day of preparation of the cosmetic. Further, since the cosmetics of Comparative Examples 1 and 5 had poor dispersibility of particles and precipitated, the viscosity of these cosmetics was not measured.

### Manufacturing Method of Cosmetic

Using the formulations shown in Tables 1 to 3, an oil-in-water type emulsified cosmetic was produced by the following method. Here, the numbers shown below correspond to the numbers on the left side indicating the ingredient names of the formulations in Tables 1 to 3.

### Example 1

After adding the materials of No. 2 to No. 4 to a part of the ion-exchanged water of No. 1 to uniformly mix, the material of No. 5, a part of the material of No. 6, and the materials of No. 7 to No. 9 were uniformly mixed to obtain an aqueous phase part.

The materials of No. 18 to No. 21 and the hydrophobically treated microparticles of No. 28 were uniformly mixed to obtain an oil phase part.

The remaining ion-exchanged water of No. 1, the remainder of No. 6, the nonionic surfactant of No. 12 and the pigment grade hydrophobically treated particles of No. 13 were uniformly mixed to obtain a powder part.

After gradual addition of the oil phase part to the aqueous phase part, a mixed liquid was prepared by gradual addition of the materials of No. 22 to No. 27. Then, a dispersion liquid in which silica of No. 11 was dispersed in ethanol of No. 10 was gradually added to this mixed liquid, and then the powder part was gradually added thereto and uniformly dispersed with a dispenser to obtain the oil-in-water type emulsified cosmetic of Example 1.

### Examples 2 to 7 and Comparative Examples 1 and 2

The oil-in-water type emulsified cosmetics of Examples 2 to 7 and Comparative Examples 1 to 2 were obtained in the same manner as in Example 1, except that the formulations were changed to those shown in Table 1.

### Comparative Example 3: A System in Which Pigment Grade Hydrophilic Particles Were Blended Into an Aqueous Phase (Outer Phase) and Hydrophobically Treated Microparticles Were Blended Into an Oil Phase (Inner Phase)

After adding the materials of No. 2 to No. 4 to a part of the ion-exchanged water of No. 1 to uniformly mix, the material of No. 5, a part of the material of No. 6, and the materials of No. 7 to No. 9 were uniformly mixed to obtain an aqueous phase part.

The materials of No. 18 to No. 21 and the hydrophobically treated microparticles of No. 28 were uniformly mixed to obtain an oil phase part.

The remaining ion-exchanged water of No. 1, the remainder of No. 6, the nonionic surfactant of No. 12 and the pigment grade hydrophilic particles of No. 17 were uniformly mixed to obtain a powder part.

After gradual addition of the oil phase part to the aqueous phase part, a mixed liquid was prepared by gradual addition of the materials of No. 22 to No. 27. Then, a dispersion liquid in which silica of No. 11 was dispersed in ethanol of No. 10 was gradually added to this mixed liquid, and then the powder part was gradually added thereto and uniformly dispersed with a dispenser to obtain the oil-in-water type emulsified cosmetic of Comparative Example 3.

### Comparative Example 4: A System in Which Pigment Grade Hydrophobically Treated Particles and Hydrophobically Treated Microparticles Were Blended Into an Oil Phase (Inner Phase)

After adding the materials of No. 2 to No. 4 to the ion-exchanged water of No. 1 to uniformly mix, the materials of No. 5 to No. 9 and No. 12 were uniformly mixed to obtain an aqueous phase part.

The materials of No. 18 to No. 21, the hydrophobically treated microparticles of No. 28 and the pigment grade hydrophobically treated particles of No. 29 were uniformly mixed to obtain an oil phase part.

After gradual addition of the oil phase part to the aqueous phase part, a mixed liquid was prepared by gradual addition of the materials of No. 22 to No. 27. Then, a dispersion liquid in which silica of No. 11 was dispersed in ethanol of No. 10 was gradually added to this mixed liquid, and uniformly dispersed with a dispenser to obtain the oil-in-water type emulsified cosmetic of Comparative Example 4.

### Comparative Example 5: A System in Which Pigment Grade Hydrophobically Treated Particles and Hydrophobically Treated Microparticles Were Blended Into an Aqueous Phase (Outer Phase)

After adding the materials of No. 2 to No. 4 to a part of the ion-exchanged water of No. 1 to uniformly mix, the material of No. 5, a part of the material of No. 6, and the materials of No. 7 to No. 9 were uniformly mixed to obtain an aqueous phase part.

The materials of No. 18 to No. 21 were uniformly mixed to obtain an oil phase part.

The remaining ion-exchanged water of No. 1, the remainder of No. 6, the nonionic surfactant of No. 12, the pigment grade hydrophobically treated particles of No. 13, and the hydrophobically treated microparticles of No. 16 were uniformly mixed to obtain a powder part.

After gradual addition of the oil phase part to the aqueous phase part, a mixed liquid was prepared by gradual addition of the materials of No. 22 to No. 27. Then, a dispersion liquid in which silica of No. 11 was dispersed in ethanol of No. 10 was gradually added to this mixed liquid, and then the powder part was gradually added thereto and uniformly dispersed with a dispenser to obtain the oil-in-water type emulsified cosmetic of Comparative Example 5.

### Examples 8 to 13 and Comparative Example 6

The oil-in-water type emulsified cosmetics of Examples 8 to 13 and Comparative Example 6 were obtained in the same manner as in Example 1, except that the formulations were changed to those shown in Table 3. Note that the oil contents of No. 24 to No. 27 were blended at the time of preparation of the oil

TABLE 1

| | | | Example | Example | Example | Example | Example |
|---|---|---|---|---|---|---|---|
| No. | Ingredients | | 1 | 2 | 3 | 4 | 5 |
| 1 | Water | Ion-exchanged water | 38.49 | 38.49 | 38.42 | 55.99 | 38.31 |
| 2 | pH adjuster | Citric acid | 0.065 | 0.065 | 0.065 | 0.065 | 0.065 |
| 3 | pH adjuster | Sodium citrate | 0.005 | 0.005 | 0.005 | 0.005 | 0.005 |
| 4 | Chelating agent | EDTA-2Na | 0.05 | 0.05 | 0.05 | 0.05 | 0.05 |
| 5 | Moisturizing agent | Glycerin | 3 | 3 | 3 | 3 | 3 |
| 6 | Polyhydric alcohol | 1,3-butylene glycol | 3.4 | 3.4 | 3.4 | 3.4 | 3.4 |
| 7 | Nonionic surfactant | PEG-60 hydrogenated | 1.3 | 1.3 | 1.3 | 1.3 | 1.3 |
| | (HLB 14.0) | castor oil | | | | | |
| 8 | Thickener | Succinoglycan | 0.12 | 0.12 | 0.12 | 0.12 | 0.12 |
| 9 | Thickener | (dimethylacrylamide/ | 0.3 | 0.3 | 0.3 | 0.3 | 0.3 |
| | | acryloyldimethyltaurine | | | | | |
| | | Na) cross polymer | | | | | |
| 10 | Lower alcohol | Ethanol | 5 | 5 | 5 | 5 | 5 |
| 11 | Usability powder | Silica | 1.5 | 1.5 | 1.5 | 1.5 | 1.5 |
| 12 | Nonionic surfactant | PEG(11) methyl | 0.07 | 0.07 | 0.14 | 0.07 | 0.25 |
| | (polyether-modified | ether dimethicone | | | | | |
| | silicone, HLB 14.5) | | | | | | |
| 13 | Pigment grade | Silicone treated | 2 | — | 2 | 2 | 2 |
| | hydrophobically | titanium oxide particles | | | | | |
| | treated | Average particle | | | | | |
| | particles | diameter of 300 nm | | | | | |
| 14 | Pigment grade | Silicone treated | — | 2 | — | — | — |
| | hydrophobically | titanium oxide particles | | | | | |
| | treated | Average particle | | | | | |
| | particles | diameter of 570 nm | | | | | |
| 15 | Hydrophobically | Silicone treated | — | — | — | — | — |
| | treated | titanium oxide particles | | | | | |
| | microparticles | Average particle | | | | | |
| | | diameter of 260 nm | | | | | |
| 16 | Hydrophobically | Silicone treated | — | — | — | — | — |
| | treated | zinc oxide particles | | | | | |
| | microparticles | Average particle | | | | | |
| | | diameter of 25 nm | | | | | |
| 17 | Pigment grade | Hydrophilic titanium | — | — | — | — | — |
| | hydrophilic | oxide particles | | | | | |
| | particles | Average particle | | | | | |
| | | diameter of 300 nm | | | | | |
| 18 | Oil content | Isododecane | 10 | 10 | 10 | 5 | 10 |
| | (volatile non-polar oil) | | | | | | |
| 19 | Oil content | Dimethicone (1.5 cst) | 7 | 7 | 7 | 4 | 7 |
| | (volatile non-polar oil) | | | | | | |
| 20 | Dispersant | Sorbitan | 0.2 | 0.2 | 0.2 | 0.2 | 0.2 |
| | | sesquiisostearate | | | | | |
| 21 | Dispersant | Isostearic acid | 0.8 | 0.8 | 0.8 | 0.8 | 0.8 |
| 22 | Ultraviolet absorber | Ethylhexyl | 7.5 | 7.5 | 7.5 | 3 | 7.5 |
| | (non-volatile polar oil) | methoxy cinnamate | | | | | |
| 23 | Ultraviolet absorber | Ethylhexyltriazone | 1 | 1 | 1 | — | 1 |
| | (non-volatile polar oil) | | | | | | |
| 24 | Ultraviolet absorber | Bisethylhexyloxyphenol | 2 | 2 | 2 | — | 2 |
| | (non-volatile polar oil) | methoxyphenyltriazine | | | | | |
| 25 | Oil content | PPG-17 | 1 | 1 | 1 | 1 | 1 |
| | (non-volatile non-polar oil) | | | | | | |
| 26 | Nonionic surfactant | PEG-100 hydrogenated | 0.2 | 0.2 | 0.2 | 0.2 | 0.2 |
| | (HLB 15.0) | castor oil | | | | | |
| 27 | Oil content | Dimethicone (6 cst) | 3 | 3 | 3 | 1 | 3 |
| | (non-volatile non-polar oil) | | | | | | |
| 28 | Hydrophobically | Silicone treated | 12 | 12 | 12 | 12 | 12 |
| | treated | zinc oxide particles | | | | | |
| | microparticles | Average particle | | | | | |
| | | diameter of 25 nm | | | | | |
| 29 | Pigment grade | Silicone treated | — | — | — | — | — |
| | hydrophobically | titanium oxide particles | | | | | |
| | treated | Average particle | | | | | |
| | particles | diameter of 300 nm | | | | | |

| Total | | 100 | 100 | 100 | 100 | 100 |
|---|---|---|---|---|---|---|
| Percentage of volatile oil in oil content (mass %) | | 54.0 | 54.0 | 54.0 | 64.3 | 54.0 |
| Mass ratio of pigment grade hydrophobically treated particles | | 29 | 29 | 14 | 29 | 8 |
| to polyether-modified silicone (nonionic surfactant) having an HLB of 14.5 | | | | | | |
| Evalu- | Tone-up property | A | A | A | B | B |
| ation | Particle dispersibility | A | A | A | A | A |
| | Rolling stability | A | B | B | B | C |
| | Usability | A | A | A | C | C |
| | Viscosity (mPa · s) | 8,220 | 9,440 | 8,890 | 2,390 | 9,020 |

| | | | Example | Example | Comparative | Comparative |
|---|---|---|---|---|---|---|
| No. | Ingredients | | 6 | 7 | Example 1 | Example 2 |
| 1 | Water | Ion-exchanged water | 38.38 | 38.49 | 38.49 | 38.49 |
| 2 | pH adjuster | Citric acid | 0.065 | 0.065 | 0.065 | 0.065 |
| 3 | pH adjuster | Sodium citrate | 0.005 | 0.005 | 0.005 | 0.005 |
| 4 | Chelating agent | EDTA-2Na | 0.05 | 0.05 | 0.05 | 0.05 |
| 5 | Moisturizing agent | Glycerin | 3 | 3 | 3 | 3 |
| 6 | Polyhydric alcohol | 1,3-butylene glycol | 3.4 | — | 3.4 | 3.4 |
| 7 | Nonionic surfactant | PEG-60 hydrogenated | 1.3 | 1.3 | 1.3 | 1.3 |
| | (HLB 14.0) | castor oil | | | | |
| 8 | Thickener | Succinoglycan | 0.12 | 0.12 | 0.12 | 0.12 |
| 9 | Thickener | (dimethylacrylamide/ | 0.3 | 0.3 | 0.3 | 0.3 |
| | | acryloyldimethyltaurine | | | | |
| | | Na) cross polymer | | | | |
| 10 | Lower alcohol | Ethanol | 5 | 5 | 5 | 5 |
| 11 | Usability powder | Silica | 1.5 | 1.5 | 1.5 | 1.5 |
| 12 | Nonionic surfactant | PEG(11) methyl | 0.18 | 0.07 | 0.07 | 0.07 |
| | (polyether-modified | ether dimethicone | | | | |
| | silicone, HLB 14.5) | | | | | |
| 13 | Pigment grade | Silicone treated | 2 | 2 | — | — |
| | hydrophobically | titanium oxide particles | | | | |
| | treated | Average particle | | | | |
| | particles | diameter of 300 nm | | | | |
| 14 | Pigment grade | Silicone treated | — | — | — | — |
| | hydrophobically | titanium oxide particles | | | | |
| | treated | Average particle | | | | |
| | particles | diameter of 570 nm | | | | |
| 15 | Hydrophobically | Silicone treated | — | — | — | 2 |
| | treated | titanium oxide particles | | | | |
| | microparticles | Average particle | | | | |
| | | diameter of 260 nm | | | | |
| 16 | Hydrophobically | Silicone treated | — | — | 2 | — |
| | treated | zinc oxide particles | | | | |
| | microparticles | Average particle | | | | |
| | | diameter of 25 nm | | | | |
| 17 | Pigment grade | Hydrophilic titanium | — | — | — | — |
| | hydrophilic | oxide particles | | | | |
| | particles | Average particle | | | | |
| | | diameter of 300 nm | | | | |
| 18 | Oil content | Isododecane | 10 | 10 | 10 | 10 |
| | (volatile non-polar oil) | | | | | |
| 19 | Oil content | Dimethicone (1.5 cst) | 7 | 7 | 7 | 7 |
| | (volatile non-polar oil) | | | | | |
| 20 | Dispersant | Sorbitan | 0.2 | 0.2 | 0.2 | 0.2 |
| | | sesquiisostearate | | | | |
| 21 | Dispersant | Isostearic acid | 0.8 | 0.8 | 0.8 | 0.8 |
| 22 | Ultraviolet absorber | Ethylhexyl | 7.5 | 7.5 | 7.5 | 7.5 |
| | (non-volatile polar oil) | methoxy cinnamate | | | | |
| 23 | Ultraviolet absorber | Ethylhexyltriazone | 1 | 1 | 1 | 1 |
| | (non-volatile polar oil) | | | | | |
| 24 | Ultraviolet absorber | Bisethylhexyloxyphenol | 2 | 2 | 2 | 2 |
| | (non-volatile polar oil) | methoxyphenyltriazine | | | | |
| 25 | Oil content | PPG-17 | 1 | 4.4 | 1 | 1 |
| | (non-volatile non-polar oil) | | | | | |
| 26 | Nonionic surfactant | PEG-100 hydrogenated | 0.2 | 0.2 | 0.2 | 0.2 |
| | (HLB 15.0) | castor oil | | | | |
| 27 | Oil content | Dimethicone (6 cst) | 3 | 3 | 3 | 3 |
| | (non-volatile non-polar oil) | | | | | |
| 28 | Hydrophobically | Silicone treated | 12 | 12 | 12 | 12 |
| | treated | zinc oxide particles | | | | |
| | microparticles | Average particle | | | | |
| | | diameter of 25 nm | | | | |
| 29 | Pigment grade | Silicone treated | — | — | — | — |
| | hydrophobically | titanium oxide particles | | | | |
| | treated | Average particle | | | | |
| | particles | diameter of 300 nm | | | | |

| Total | | 100 | 100 | 100 | 100 |
|---|---|---|---|---|---|
| Percentage of volatile oil in oil content (mass %) | | 54.0 | 48.7 | 54.0 | 54.0 |
| Mass ratio of pigment grade hydrophobically treated particles | | 11 | 29 | 29 | 29 |
| to polyether-modified silicone (nonionic surfactant) having an HLB of 14.5 | | | | | |
| Evalu- | Tone-up property | B | B | C | C |
| ation | Particle dispersibility | A | A | C | B |
| | Rolling stability | C | C | D | C |
| | Usability | B | C | C | B |
| | Viscosity (mPa · s) | 9,170 | 9,330 | Unmeasured | 7,710 |

### Results

As is apparent from Table 1, the cosmetics of Examples 1 to 7 containing the pigment grade hydrophobically treated particles in the aqueous phase, the hydrophobically treated microparticles in the oil phase, and the volatile oil as the oil content exhibited excellent tone-up performance, and therefore, it was confirmed that these cosmetics could uniformly apply an oil film containing the pigment grade hydrophobically treated particles, the hydrophobically treated microparticles, and the ultraviolet absorber (oil content) to the skin surface.

When comparing the cosmetics of Example 1 and Example 5, it was found that the cosmetic of Example 1, in which the mass ratio of pigment grade hydrophobically treated particles to polyether modified silicone which is a nonionic surfactant was 29, which is greater than 8, had better tone-up property and rolling stability and was less prone to cause defects related to usability, especially sticky feeling. Further, even when comparing the cosmetics of Example 5 and Example 6, it was found that the cosmetic of Example 6, in which the mass ratio of pigment grade hydrophobically treated particles to polyether modified silicone was 11, which is greater than 8, was less prone to cause defects related to sticky feeling. This sticky feeling is considered to be influenced by an increase in the amount of the nonionic surfactant used.

As can be seen from the results of Example 7, it could be confirmed that even polyhydric alcohols other than 1,3-butylene glycol were excellent in performance such as tone-up property.

On the other hand, in the cosmetic of Comparative Example 1, in which hydrophobically treated microparticles having an average particle diameter of 100 nm, which were smaller in particle diameter than pigment grade hydrophobically treated particles, were contained in an aqueous phase, particles precipitated and good tone-up property and usability could not be obtained.

As is apparent from comparing the cosmetic in Comparative Example 2, in which hydrophobically treated microparticles having an average particle diameter of 260 nm, which were smaller in particle diameter than pigment grade hydrophobically treated particles, were contained in an aqueous phase, with the cosmetic in Example 1, in which pigment grade hydrophobically treated particles having an average particle diameter of 300 nm were contained in an aqueous phase, it was found that the cosmetic of Comparative Example 2 was inferior in all performances of tone-up property, particle dispersibility, rolling stability, and usability.

TABLE 2

| | | | Comparative | Comparative | Comparative |
|---|---|---|---|---|---|
| No. | Ingredients | | Example 3 | Example 4 | Example 5 |
| 1 | Water | Ion-exchanged water | 38.49 | 38.49 | 38.49 |
| 2 | pH adjuster | Citric acid | 0.065 | 0.065 | 0.065 |
| 3 | pH adjuster | Sodium citrate | 0.005 | 0.005 | 0.005 |
| 4 | Chelating agent | EDTA-2Na | 0.05 | 0.05 | 0.05 |
| 5 | Moisturizing agent | Glycerin | 3 | 3 | 3 |
| 6 | Polyhydric alcohol | 1,3-butylene glycol | 3.4 | 3.4 | 3.4 |
| 7 | Nonionic surfactant | PEG-60 hydrogenated | 1.3 | 1.3 | 1.3 |
| | (HLB 14.0) | castor oil | | | |
| 8 | Thickener | Succinoglycan | 0.12 | 0.12 | 0.12 |
| 9 | Thickener | (dimethylacrylamide/ | 0.3 | 0.3 | 0.3 |
| | | acryloyldimethyltaurine NA) | | | |
| | | cross polymer | | | |
| 10 | Lower alcohol | Ethanol | 5 | 5 | 5 |
| 11 | Usability powder | Silica | 1.5 | 1.5 | 1.5 |
| 12 | Nonionic surfactant (polyether- | PEG(11) methyl ether dimethicone | 0.07 | 0.07 | 0.07 |
| | modified silicone, HLB 14.5) | | | | |
| 13 | Pigment grade hydrophobically | Silicone treated titanium oxide particles | — | — | 2 |
| | treated particles | Average particle diameter of 300 nm | | | |
| 14 | Pigment grade hydrophobically | Silicone treated titanium oxide particles | — | — | — |
| | treated particles | Average particle diameter of 570 nm | | | |
| 15 | Hydrophobically treated | Silicone treated titanium oxide particles | — | — | — |
| | microparticles | Average particle diameter of 260 nm | | | |
| 16 | Hydrophobically treated | Silicone treated zinc oxide particles | — | — | 12 |
| | microparticles | Average particle diameter of 25 nm | | | |
| 17 | Pigment grade hydrophilic | Hydrophilic titanium oxide particles | 2 | — | — |
| | particles | Average particle diameter of 300 nm | | | |
| 18 | Oil content (volatile | Isododecane | 10 | 10 | 10 |
| | non-polar oil) | | | | |
| 19 | Oil content (volatile | Dimethicone (1.5 cst) | 7 | 7 | 7 |
| | non-polar oil) | | | | |
| 20 | Dispersant | Sorbitan sesquiisostearate | 0.2 | 0.2 | 0.2 |
| 21 | Dispersant | Isostearic acid | 0.8 | 0.8 | 0.8 |
| 22 | Ultraviolet absorber (polar oil) | Ethylhexyl methoxy cinnamate | 7.5 | 7.5 | 7.5 |
| 23 | Ultraviolet absorber (polar oil) | Ethylhexyltriazone | 1 | 1 | 1 |
| 24 | Ultraviolet absorber (polar oil) | Bisethylhexyloxyphenol | 2 | 2 | 2 |
| | | methoxyphenyltriazine | | | |
| 25 | Oil content (non-volatile | PPG-17 | 1 | 1 | 1 |
| | non-polar oil) | | | | |
| 26 | Nonionic surfactant (HLB 15.0) | PEG-100 hydrogenated castor oil | 0.2 | 0.2 | 0.2 |
| 27 | Oil content (non-volatile | Dimethicone (6 cst) | 3 | 3 | 3 |
| | non-polar oil) | | | | |
| 28 | Hydrophobically treated | Silicone treated zinc oxide particles | 12 | 12 | — |
| | microparticles | Average particle diameter of 25 nm | | | |
| 29 | Pigment grade hydrophobically | Silicone treated titanium oxide particles | — | 2 | — |
| | treated particles | Average particle diameter of 300 nm | | | |

| Total | | 100 | 100 | 100 |
|---|---|---|---|---|
| Percentage of volatile oil in oil content (mass %) | | 54.0 | 54.0 | 54.0 |
| Mass ratio of pigment grade particles to polyether-modified | | 29 | 29 | 29 |
| silicone (nonionic surfactant) having an HLB of 14.5 | | | | |
| Evaluation | Tone-up property | C | C | C |
| | Particle dispersibility | B | B | C |
| | Rolling stability | C | C | D |
| | Usability | C | C | C |
| | Viscosity (mPa · s) | 13,370 | 8,910 | Unmeasured |

### Results

The cosmetic of Comparative Example 3 in which the pigment grade hydrophilic particles were blended in the aqueous phase (outer phase), and the hydrophobically treated microparticles were blended in the oil phase (inner phase), as shown in FIG. 2, the cosmetic of Comparative Example 4 in which the pigment grade hydrophobically treated particles and the hydrophobically treated microparticles were blended in the oil phase (inner phase), as shown in FIG. 3, and the cosmetic of Comparative Example 5 in which the pigment grade hydrophobically treated particles and the hydrophobically treated microparticles were blended in the aqueous phase (outer phase), as shown in FIG. 4 could not obtain good tone-up property.

TABLE 3

| | | | Example | Example | Example | Example | Example |
|---|---|---|---|---|---|---|---|
| No. | Ingredients | | 1 | 8 | 9 | 10 | 11 |
| 1 | Water | Ion-exchanged water | 38.49 | 38.49 | 37.46 | 38.49 | 45.38 |
| 2 | pH adjuster | Citric acid | 0.065 | 0.065 | 0.065 | 0.065 | 0.05 |
| 3 | pH adjuster | Sodium citrate | 0.005 | 0.005 | 0.005 | 0.005 | 0.0001 |
| 4 | Chelating | EDTA-2Na | 0.05 | 0.05 | 0.05 | 0.05 | 0.05 |
| | agent | | | | | | |
| 5 | Moisturizing | Glycerin | 3 | 3 | 3 | 3 | 2 |
| | agent | | | | | | |
| 6 | Polyhydric | 1,3-butylene glycol | 3.4 | 3.4 | 3.4 | 3.4 | 7 |
| | alcohol | | | | | | |
| 7 | Nonionic | PEG-60 hydrogenated | 1.3 | 1.3 | 1.3 | 1.3 | — |
| | surfactant (HLB 14.0) | castor oil | | | | | |
| 8 | Nonionic | PEG-40 hydrogenated | — | — | — | — | 0.5 |
| | surfactant (HLB 12.0) | castor oil | | | | | |
| 9 | Thickener | Succinoglycan | 0.12 | 0.12 | 0.12 | 0.12 | — |
| 10 | Thickener | (dimethylacrylamide/ | 0.3 | 0.3 | 0.3 | 0.3 | 0.6 |
| | | acrylobyldimethyltaurine | | | | | |
| | | Na) cross polymer | | | | | |
| 11 | Thickener | Stearoxyhydroxypropyl | — | — | — | — | 0.02 |
| | | methylcellulose | | | | | |
| 12 | Lower alcohol | Ethanol | 5 | 5 | 5 | 5 | 7.5 |
| 13 | Usability powder | Silica | 1.5 | 1.5 | 1.5 | 1.5 | — |
| 14 | Nonionic surfactant | PEG(11) methyl | 0.07 | 0.07 | 0.1 | 0.07 | 0.3 |
| | (polyether-modified | ether dimethicone | | | | | |
| | silicone, HLB 14.5) | | | | | | |
| 15 | Pigment grade | Silicone treated | 2 | — | 2 | 2 | — |
| | hydrophobically | titanium oxide particles | | | | | |
| | treated particles | Average particle | | | | | |
| | | diameter of 300 nm | | | | | |
| 16 | Pigment grade | Silicone treated | — | 2 | 1 | — | — |
| | hydrophobically | titanium oxide coated | | | | | |
| | treated particles | mica particles | | | | | |
| | (pearl agent) | Average particle | | | | | |
| | | diameter of 22 μm | | | | | |
| 17 | Pigment grade | Silicone treated | — | — | — | — | 2 |
| | hydrophobically | titanium oxide particles | | | | | |
| | treated particles | Average particle | | | | | |
| | | diameter of 500 nm | | | | | |
| 18 | Pigment grade | Silicone treated titanium | — | — | — | — | 2 |
| | hydrophobically | oxide-silica coated | | | | | |
| | treated particles | mica particles | | | | | |
| | (pearl agent) | Average particle diameter | | | | | |
| | | of 30 μm | | | | | |
| 19 | Pigment grade | Silicone treated titanium | — | — | — | — | 1 |
| | hydrophobically | oxide-silica coated | | | | | |
| | treated particles | mica particles | | | | | |
| | (pearl agent) | Average particle diameter | | | | | |
| | | of 30 μm | | | | | |
| 20 | Pigment grade | Silicone treated titanium | — | — | — | — | 1 |
| | hydrophobically | oxide coated mica particles | | | | | |
| | treated particles | Average particle diameter | | | | | |
| | (pearl agent) | of 5 μm | | | | | |
| 21 | Oil content | Isododecane | 10 | 10 | 10 | 5 | — |
| | (volatile non-polar oil) | | | | | | |
| 22 | Oil content | Dimethicone | 7 | 7 | 7 | 3.5 | — |
| | (volatile non-polar oil) | (1.5 cst) | | | | | |
| 23 | Oil content | Dimethicone | — | — | — | — | 3 |
| | (volatile non-polar oil) | (2 cst) | | | | | |
| 24 | Oil content | Dimethicone | — | — | — | — | 1 |
| | (non-volatile non-polar oil) | (20 cst) | | | | | |
| 25 | Oil content | Isopropyl | — | — | — | 2 | — |
| | (non-volatile polar oil) | myristate | | | | | |
| 26 | Oil content | Triethylhexanoin | — | — | — | 2.5 | — |
| | (non-volatile polar oil) | | | | | | |
| 27 | Oil content | Diphenylsiloxyphenyl | — | — | — | 4 | 2 |
| | (non-volatile non-polar oil) | trimethicone | | | | | |
| 28 | Dispersant | Sorbitan | 0.2 | 0.2 | 0.2 | 0.2 | — |
| | | sesquiisostearate | | | | | |
| 29 | Dispersant | Isostearte acid | 0.8 | 0.8 | 0.8 | 0.8 | — |
| 30 | Ultraviolet | Ethylhexyl methoxy | 7.5 | 7.5 | 7.5 | 7.5 | 7 |
| | absorber (polar oil) | cinnamate | | | | | |
| 31 | Ultraviolet | Ethylhexyltriazone | 1 | 1 | 1 | 1 | 0.5 |
| | absorber (polar oil) | | | | | | |
| 32 | Ultraviolet | Bisethylhexyloxyphenol | 2 | 2 | 2 | 2 | 2 |
| | absorber (polar oil) | methoxyphenyltriazine | | | | | |
| 33 | Ultraviolet | Hexyl | — | — | — | — | 2 |
| | absorber (polar oil) | diethylamino | | | | | |
| | | hydroxybenzoybenzoate | | | | | |
| 34 | Ultraviolet | Homosalate | — | — | — | — | 1.6 |
| | absorber (polar oil) | | | | | | |
| 35 | Oil content | PPG-17 | 1 | 1 | 1 | 1 | — |
| | (non-volatile non-polar oil) | | | | | | |
| 36 | Oil content | Polybutylene glycol/ | — | — | — | — | 1 |
| | (non-volatile non-polar oil) | PPG-9/1 copolymer | | | | | |
| 37 | Nonionic surfactant | PEG-12 dimethicone | — | — | — | — | 1.5 |
| | (polyether-modified | | | | | | |
| | silicone, HLB 8.0) | | | | | | |
| 38 | Nonionic surfactant | PEG-100 hydrogenated | 0.2 | 0.2 | 0.2 | 0.2 | — |
| | (HLB 15.0) | castor oil | | | | | |
| 39 | Oil content | Dimethicone (6 cst) | 3 | 3 | 3 | 3 | — |
| | (non-volatile non-polar oil) | | | | | | |
| 40 | Hydrophobically | Silicone treated zinc | 12 | 12 | 12 | 12 | 9 |
| | treated | oxide particles, average | | | | | |
| | microparticles | particle diameter of 25 nm | | | | | |

| Total | | 100 | 100 | 100 | 100 | 100 |
|---|---|---|---|---|---|---|
| Percentage of volatile oil in oil content (mass %) | | 54.0 | 54.0 | 54.0 | 27.0 | 14.9 |
| Mass ratio of pigment grade particles to polyether-modified | | 29 | 29 | 30 | 29 | 20 |
| silicone (nonionic surfactant) having an HLB of 14.5 | | | | | | |
| Evalu- | Tone-up property | A | B | A | B | B |
| ation | Particle dispersibility | A | A | A | A | A |
| | Rolling stability | A | C | C | B | A |
| | Usability | A | A | A | B | A |
| | Viscosity (mPa · s) | 8,220 | 5,820 | 3,150 | 3,860 | 5,710 |

| | | | Example | Example | Comparative |
|---|---|---|---|---|---|
| No. | Ingredients | | 12 | 13 | Example 6 |
| 1 | Water | Ion-exchanged water | 45.38 | 43.97 | 38.49 |
| 2 | pH adjuster | Citric acid | 0.05 | 0.05 | 0.065 |
| 3 | pH adjuster | Sodium citrate | 0.0001 | 0.0001 | 0.005 |
| 4 | Chelating | EDTA-2Na | 0.05 | 0.05 | 0.05 |
| | agent | | | | |
| 5 | Moisturizing | Glycerin | 2 | 2 | 3 |
| | agent | | | | |
| 6 | Polyhydric | 1,3-butylene glycol | 7 | 5 | 3.4 |
| | alcohol | | | | |
| 7 | Nonionic | PEG-60 hydrogenated | — | — | 1.3 |
| | surfactant (HLB 14.0) | castor oil | | | |
| 8 | Nonionic | PEG-40 hydrogenated | 0.5 | 0.5 | — |
| | surfactant (HLB 12.0) | castor oil | | | |
| 9 | Thickener | Succinoglycan | — | — | 0.12 |
| 10 | Thickener | (dimethylacrylamide/ | 0.6 | 0.6 | 0.3 |
| | | acrylobyldimethyltaurine | | | |
| | | Na) cross polymer | | | |
| 11 | Thickener | Stearoxyhydroxypropyl | 0.02 | 0.02 | — |
| | | methylcellulose | | | |
| 12 | Lower alcohol | Ethanol | 7.5 | 7.5 | 5 |
| 13 | Usability powder | Silica | — | — | 1.5 |
| 14 | Nonionic surfactant | PEG(11) methyl | 0.3 | 0.21 | 0.07 |
| | (polyether-modified | ether dimethicone | | | |
| | silicone, HLB 14.5) | | | | |
| 15 | Pigment grade | Silicone treated | — | 2 | 2 |
| | hydrophobically | titanium oxide particles | | | |
| | treated particles | Average particle | | | |
| | | diameter of 300 nm | | | |
| 16 | Pigment grade | Silicone treated | — | — | — |
| | hydrophobically | titanium oxide coated | | | |
| | treated particles | mica particles | | | |
| | (pearl agent) | Average particle | | | |
| | | diameter of 22 μm | | | |
| 17 | Pigment grade | Silicone treated | — | — | — |
| | hydrophobically | titanium oxide particles | | | |
| | treated particles | Average particle | | | |
| | | diameter of 500 nm | | | |
| 18 | Pigment grade | Silicone treated titanium | 3 | 2 | — |
| | hydrophobically | oxide-silica coated | | | |
| | treated particles | mica particles | | | |
| | (pearl agent) | Average particle diameter | | | |
| | | of 30 μm | | | |
| 19 | Pigment grade | Silicone treated titanium | 2 | 1 | — |
| | hydrophobically | oxide-silica coated | | | |
| | treated particles | mica particles | | | |
| | (pearl agent) | Average particle diameter | | | |
| | | of 30 μm | | | |
| 20 | Pigment grade | Silicone treated titanium | 1 | 1 | — |
| | hydrophobically | oxide coated mica particles | | | |
| | treated particles | Average particle diameter | | | |
| | (pearl agent) | of 5 μm | | | |
| 21 | Oil content | Isododecane | — | — | — |
| | (volatile non-polar oil) | | | | |
| 22 | Oil content | Dimethicone | — | 12 | — |
| | (volatile non-polar oil) | (1.5 cst) | | | |
| 23 | Oil content | Dimethicone | 3 | — | — |
| | (volatile non-polar oil) | (2 cst) | | | |
| 24 | Oil content | Dimethicone | 1 | — | — |
| | (non-volatile non-polar oil) | (20 cst) | | | |
| 25 | Oil content | Isopropyl | — | — | 4 |
| | (non-volatile polar oil) | myristate | | | |
| 26 | Oil content | Triethylhexanoin | — | — | 5 |
| | (non-volatile polar oil) | | | | |
| 27 | Oil content | Diphenylsiloxyphenyl | 2 | — | 8 |
| | (non-volatile non-polar oil) | trimethicone | | | |
| 28 | Dispersant | Sorbitan | — | — | 0.2 |
| | | sesquiisostearate | | | |
| 29 | Dispersant | Isostearte acid | — | — | 0.8 |
| 30 | Ultraviolet | Ethylhexyl methoxy | 7 | 7 | 7.5 |
| | absorber (polar oil) | cinnamate | | | |
| 31 | Ultraviolet | Ethylhexyltriazone | 0.5 | — | 1 |
| | absorber (polar oil) | | | | |
| 32 | Ultraviolet | Bisethylhexyloxyphenol | 2 | 2 | 2 |
| | absorber (polar oil) | methoxyphenyltriazine | | | |
| 33 | Ultraviolet | Hexyl | 2 | — | — |
| | absorber (polar oil) | diethylaminohydroxybenzoybenzoate | | | |
| 34 | Ultraviolet | Homosalate | 1.6 | 1.6 | — |
| | absorber (polar oil) | | | | |
| 35 | Oil content | PPG-17 | — | 1 | 1 |
| | (non-volatile non-polar oil) | | | | |
| 36 | Oil content | Polybutylene glycol/ | 1 | — | — |
| | (non-volatile non-polar oil) | PPG-9/1 copolymer | | | |
| 37 | Nonionic surfactant | PEG-12 dimethicone | 1.5 | 1.5 | — |
| | (polyether-modified | | | | |
| | silicone, HLB 8.0) | | | | |
| 38 | Nonionic surfactant | PEG-100 hydrogenated | — | — | 0.2 |
| | (HLB 15.0) | castor oil | | | |
| 39 | Oil content | Dimethicone (6 cst) | — | — | 3 |
| | (non-volatile non-polar oil) | | | | |
| 40 | Hydrophobically | Silicone treated zinc | 9 | 9 | 12 |
| | treated | oxide particles, average | | | |
| | microparticles | particle diameter of 25 nm | | | |

| Total | | 100 | 100 | 100 |
|---|---|---|---|---|
| Percentage of volatile oil in oil content (mass %) | | 14.9 | 50.8 | 0 |
| Mass ratio of pigment grade particles to polyether-modified | | 20 | 29 | 29 |
| silicone (nonionic surfactant) having an HLB of 14.5 | | | | |
| Evalu- | Tone-up property | B | A | C |
| ation | Particle dispersibility | B | A | B |
| | Rolling stability | B | A | C |
| | Usability | A | A | C |
| | Viscosity (mPa · s) | 6,200 | 6,830 | 5,660 |

### Results

As is apparent from the results of Comparative Example 6, it could be confirmed that even if a cosmetic containing only non-volatile oil but not containing volatile oil was applied to the skin and the moisture was dried, the particles moved and the particles were not uniformly applied to the skin, so that a good tone-up effect could not be obtained. On the other hand, it was found that a cosmetic containing volatile oil could obtain a good tone-up effect regardless of the type of pigment grade hydrophobically treated particles.

As is apparent from comparison of Example 1 and Example 10, it was found that when the proportion of volatile oil in the oil content increased, tone-up property, rolling stability, and usability were improved. Similar trends were shown in Examples 11 and 13, and it could be confirmed that, even in a formulation containing a pearl agent, when the proportion of volatile oil in the oil content increased, tone-up property was improved.

As is apparent from comparison of Example 8 and Example 9, it was found that tone-up property was improved when a pearl agent and inorganic oxide particles such as hydrophobically treated titanium oxide particles were used in combination as the pigment grade hydrophobically treated particles than when only the pearl agent was used.

### REFERENCE SIGNS LIST

10 Water

20 Oil content

30 Hydrophobically treated microparticle

40 Pigment grade hydrophobically treated particle

45 Hydrophilic pigment grade particle

50 Skin

1. An oil-in-water type emulsified cosmetic comprising:
a dispersion medium comprising water and
an oil droplet dispersed in the dispersion medium,
wherein the dispersion medium comprises a nonionic surfactant, a polyhydric alcohol, and a pigment grade hydrophobically treated particle having an average particle diameter of 300 nm or more,
wherein the oil droplet comprises an oil content, a nonionic surfactant, and a hydrophobically treated microparticle having an average particle diameter of 200 nm or less, and
wherein the oil content comprises a volatile oil.

2. The cosmetic according to claim 1, wherein the content of the oil content is 20% by mass or more.

3. The cosmetic according to claim 1, wherein the volatile oil is contained in the oil content in an amount of 10% by mass or more.

4. The cosmetic according to claim 1, wherein an HLB of the nonionic surfactant is 10.0 to 16.0.

5. The cosmetic according to claim 1, wherein the nonionic surfactant is a polyether-modified silicone.

6. The cosmetic according to claim 5, wherein the mass ratio of the pigment grade hydrophobically treated particle to the polyether-modified silicone having an HLB of 10.0 to 16.0 is 11 to 30.

7. The cosmetic according to claim 1, wherein the oil content comprises at least one selected from a polar oil and an ultraviolet absorber.

8. The cosmetic according to claim 7, wherein the at least one selected from the polar oil and the ultraviolet absorber is contained in an amount of 10% by mass or more based on the total amount of the oil content.

9. The cosmetic according to claim 1, wherein the hydrophobically treated microparticle is an ultraviolet scattering agent.
